(19) United States
(12) Patent Application Publication
(10) Pub. No.: US 2023/0357763 A1
(43) Pub. Date: Nov. 9, 2023

(54) Morpholinos with Increased Delivery Efficiency

(71) Applicants: James E. Summerton, Corvallis, OR (US); Yongfu Li, Corvallis, OR (US); Shan Jiang, Corvallis, OR (US); Daniel Bryce Summerton, Corvallis, OR (US)

(72) Inventors: James E. Summerton, Corvallis, OR (US); Yongfu Li, Corvallis, OR (US); Shan Jiang, Corvallis, OR (US); Daniel Bryce Summerton, Corvallis, OR (US)

(21) Appl. No.: 17/803,316

(22) Filed: May 6, 2022

(51) Int. Cl. C12N 15/113

(52) U.S. Cl. CPC C12N 15/113; C12N 2310/11; C12N 2310/3233; C12N 2310/3513; C12N 2310/51; C12N 2320/30

(57) ABSTRACT

Morpholino antisense oligos (Morpholinos) are a class of synthetic non-ionic molecules, each designed to very specifically bind to a selected complementary RNA sequence (targeted RNA transcript). Custom-sequence Morpholinos are used in a broad range of biological research areas, as well as for therapeutic applications in vivo (in living animals such as humans).

For most in vivo applications the “bare” Morpholino (FIG. 1a) is linked to a cationic delivery component to give a “delivery-enabled” Morpholino (FIG. 1b) with significantly improved delivery efficiency. However, cytosolic delivery was still markedly less than expected. Then recently we discovered that by adding a special disconnect component between the Morpholino component and the cationic delivery component (FIG. 1c), the cytosolic delivery efficiency for the Morpholino was dramatically increased (about a 1,000% increase in delivery efficiency).

This patent application describes designing, making, and using such delivery-enabled Morpholinos containing a key disconnect component.

### FIELD OF THE INVENTION

The invention disclosed and claimed herein relates to designing, making, and using a Morpholino antisense oligo which is linked to a disconnect component that is, in turn, linked to a cationic delivery component.

### BACKGROUND OF THE INVENTION

Morpholino antisense oligos were devised by Summerton in 1985 and optimized from 1985 through 1989. The structure of Morpholinos is radically different from conventional nucleic-acid-based antisense oligonucleotides. Specifically, most conventional antisense oligonucleotides contain 5-membered ribose or deoxyribose backbone ring structures joined by negatively-charged inter-subunit linkages. In sharp contrast, Morpholinos uniquely contain 6-membered morpholine backbone ring structures joined by non-ionic inter-subunit linkages (see U.S. Pat. No. 5,185,444, issued 1993).

Morpholinos' novel structural elements provide outstanding properties in comparison to more conventional antiscnse agents. Specifically, Morpholinos:
a) are resistant to degradation in biological systems (including in acidified lysosomes); b) provide by far the greatest sequence specificity of all antisense structural types; c) do not require RNase H or RISC to function; d) are generally free of the non-antisense off-target effects that plague most antisense structural types; e) provide predictable targeting of one's selected RNA transcript; f) freely pass between cytosol and nucleus of cells and functions in both; are versatile g) can alter splicing in the nucleus, h) block protein translation in the cytosol, and i) block binding of regulatory proteins and non-coding RNAs throughout the cell); j) have good aqueous solubility; and, k) are affordable due to cheap starting materials, efficient assembly, and easy workup.

IN VIVO: While Morpholinos provide many key advantages over other antisense structural types, nonetheless in the context of use in vivo, and particularly for therapeutic applications, it turned out that delivery of Morpholinos from a subject's circulatory system to the cytosolic compartment of the subject's cells was quite difficult. Accordingly, considerable time and resources were expended in efforts to develop an acceptable delivery system for in vivo applications of Morpholinos—ultimately leading to a cationic delivery component that can be attached to any Morpholino and is effective to deliver the Morpholino from the circulatory system to the cytosol of most cells in most subjects (from mice to humans). Those delivery-enabled Morpholinos (devised by Li in 2007, FIG. 1b) are detailed in U.S. Pat. No. 7,935,816, issued in 2011.

While such delivery-enabled Morpholinos have been used by many research scientists around the world for more than a decade, that delivery component is less than ideal for therapeutic use in humans because: a) the cationic delivery component is somewhat toxic at higher concentrations; b) delivery efficiency is much less than desired for therapeutic applications in humans; and, c) production costs are relatively high. Thus, for a number of years efforts have been expended in attempts to develop an in vivo delivery system that would offer: a) little or no toxicity; b) a substantially increased cytosolic delivery efficiency; and, c) significantly reduced production costs.

Recently (after many failures) these long-running efforts to develop a better delivery system finally paid off with the invention described and claimed in this patent application.

### SUMMARY OF THE INVENTION

FIG. 1 illustrates the time progression of Morpholino antisense structures and their approximate respective cytosolic delivery efficiencies.

The current invention disclosed and claimed in the current patent application (FIG. 1c) closely resembles the prior-art products (shown in FIG. 1b and detailed in U.S. Pat. No. 7,935,816). That resemblance is by virtue of each product containing: 1) a custom-sequence Morpholino antisense oligo that serves to very specifically bind a selected complementary RNA sequence; and, 2) a cationic delivery component that serves to deliver the Morpholino to the cytosol of cells in a treated subject.

In sharp contrast to the above similarities, the new invention differs from prior art by incorporation of a disconnect component (FIG. 1c) that increases delivery efficiency about 1,000% compared to the prior art (FIG. 2).

It is noteworthy that such a large increase in delivery efficiency can translate to about a 600% to 800% cost reduction in the current very high cost of treating serious diseases, such as muscular dystrophy (where FDA-approved “bare” Morpholino antisense oligos (FIG. 1a) currently cost $300,000 per muscular dystrophy patient per year). Similar cost reductions are expected to soon apply to treating other serious diseases—including particularly BRCA1-defective and triple-negative breast cancers, which are expected to soon be both curable (without harming the patient) and affordable with the new advanced Morpholinos incorporating the disconnect component. Further, the affordability that comes from high delivery efficiency, achieved by incorporating the disconnect component, may prove to be an essential factor enabling wide use of Morpholinos for ending the covid-19 pandemic (functional product expected to be completed in April of 2022).

### BRIEF DESCRIPTION OF THE FIGURES

FIG. 1 illustrates the evolution of Morpholinos from “bare” Morpholinos not less than 15 and not more than 40 nucleobase subunits long (FIG. 1a) to the delivery-enabled version (FIG. 1b) currently marketed as “Vivo-Morpholinos”. FIG. 1c shows our new (yet-to-be-disclosed) version of Morpholinos with greatly improved cytosolic delivery efficiency by virtue of a disconnect component covalently inserted between the Morpholino component and the cationic dendrimer delivery component containing at least 6 positive charges and not more than 12 positive charges.

FIG. 2 shows experimental results comparing cytosolic delivery efficiencies for a delivery-enabled Morpholino lacking a disconnect component (as in FIG. 1b) versus the same delivery-enabled Morpholino—but with a disconnect component covalently inserted between the Morpholino component and the cationic delivery component (FIG. 1c).

FIG. 3 shows a synthesis scheme for a 4-amino-acid-long disconnect peptide structured for easy covalent insertion between the Morpholino component and the cationic delivery component—where that short peptide is explicitly designed to be rapidly cleaved in an acidified lysosome. Tfa protected lysine unit can be converted into homoarginine (hR) in the final step of guanidination, giving the sequence of the disconnect peptide: Val-hR-Gly-Gly. Alternatively, orthogonally protected lysine can keep the lysine unchanged, giving the sequence of the disconnect peptide: Val-Lys-Gly-Gly.

FIG. 4 shows a synthesis scheme for an 8-amino-acid-long disconnect peptide structured for easy covalent insertion between the Morpholino component and the cationic delivery component—where that longer peptide is explicitly designed to be rapidly cleaved in an acidified lysosome.

FIG. 5 shows the preferred sites where the disconnect components of FIGS. 3 and 4 are cleaved within acidified lysosomes.

FIGS. 6A and 6B show a representative synthesis scheme by which a disconnect peptide structure, such as in FIGS. 3 and 4, can be covalently inserted between a Morpholino component and a cationic delivery component in order to give a high-delivery-efficiency delivery-enabled Morpholino with disconnect component, with the general structure shown in FIG. 1c.

FIG. 7 shows the two fragments resulting from cleavage of the disconnect component shown in FIG. 3, where such disconnection occurs within acidified lysosomes.

### DETAILED DESCRIPTION OF INVENTION

Experimental results from the 1990s suggest there is good reason to believe that our delivery-enabled Morpholinos (U.S. Pat. No. 7,935,816) achieve much less cytosolic delivery than should be possible. To remedy this postulated under-achievement of cytosolic delivery, years ago we set out to look for an explanation for what appears to be substantial under-achievement of cytosolic delivery. The hope was that if we could understand the cause of that poor delivery then we might be able to remedy that poor delivery.

After many failed searches, a promising postulate was finally devised that appeared to account for low delivery efficiency of delivery-enabled Morpholinos. The postulate was that the cationic delivery component (which partially permeabilizes lysosomal membranes) will preferentially remain bound to the inner surface of the lysosome. Then, because the Morpholino component is covalently linked to the cationic delivery component, that connection may act to substantially impede passage of the Morpholino component through the partially permeabilized lysosomal membrane and on into the cytosol/nuclear compartment of the cell—wherein it was intended that the delivered Morpholino would then find and block its complementary targeted RNA sequence.

If the above is indeed the case, then it was further postulated that the low delivery efficiency problem might be fixed by inserting a special link between the Morpholino component and the cationic delivery component, where that link is explicitly designed to be rapidly cleaved ONLY upon acidification in the lysosome (a natural process). Cleavage of that special link would thereby allow the newly-disconnected Morpholino component (which is very small in two dimensions) to more rapidly pass through the partially permeabilized lysosomal membrane and on into the cytosol/nuclear compartment of the cell (Morpholinos are known to move freely between cytosol and nucleus).

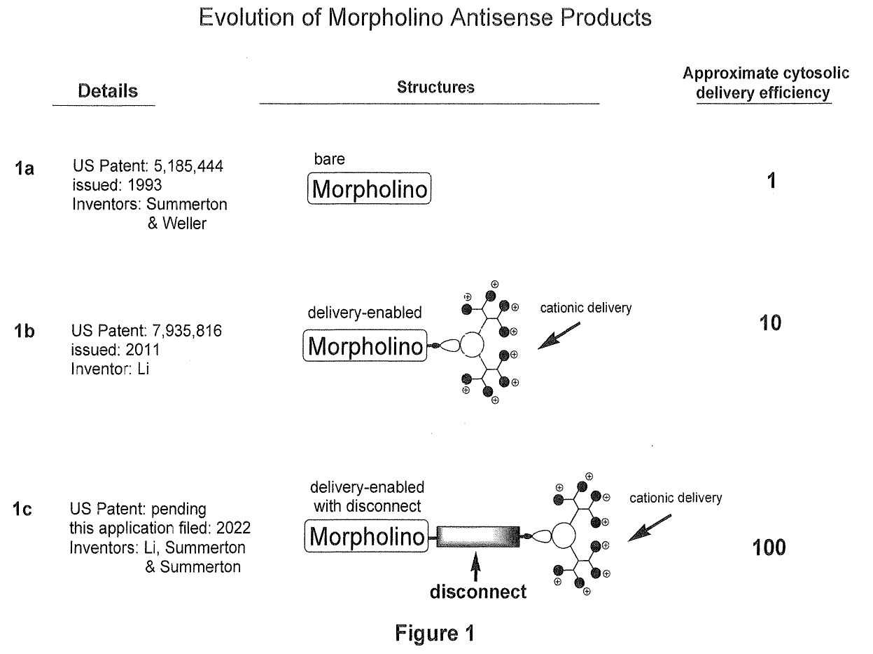

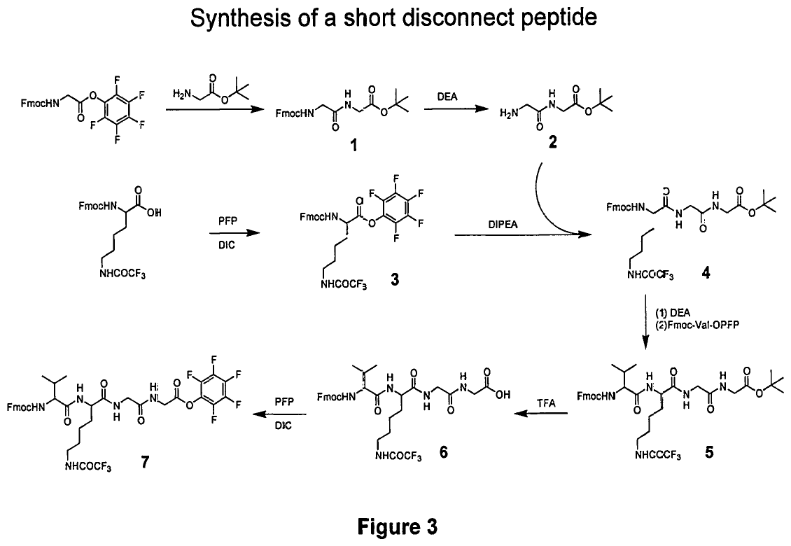

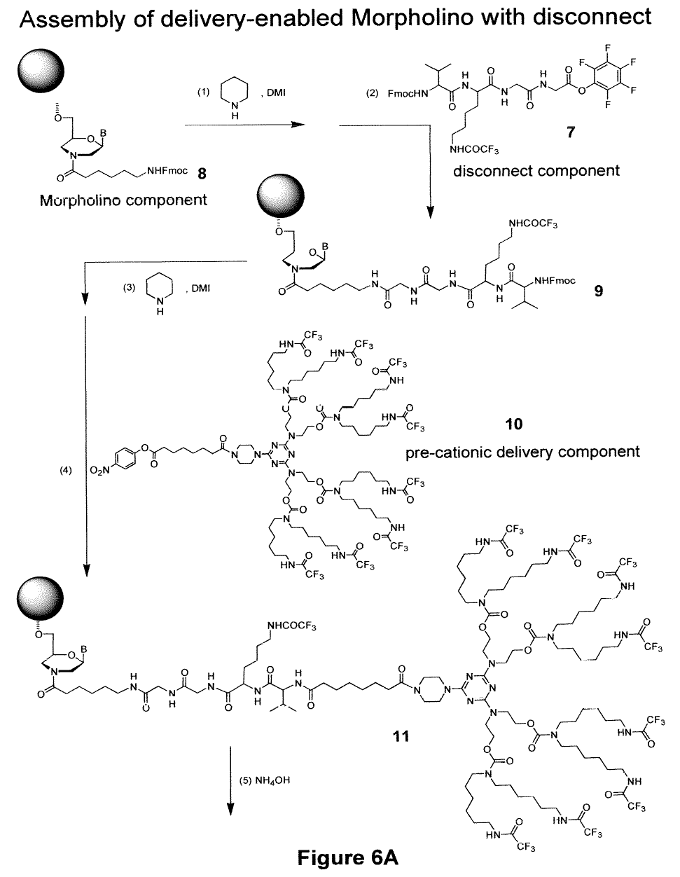

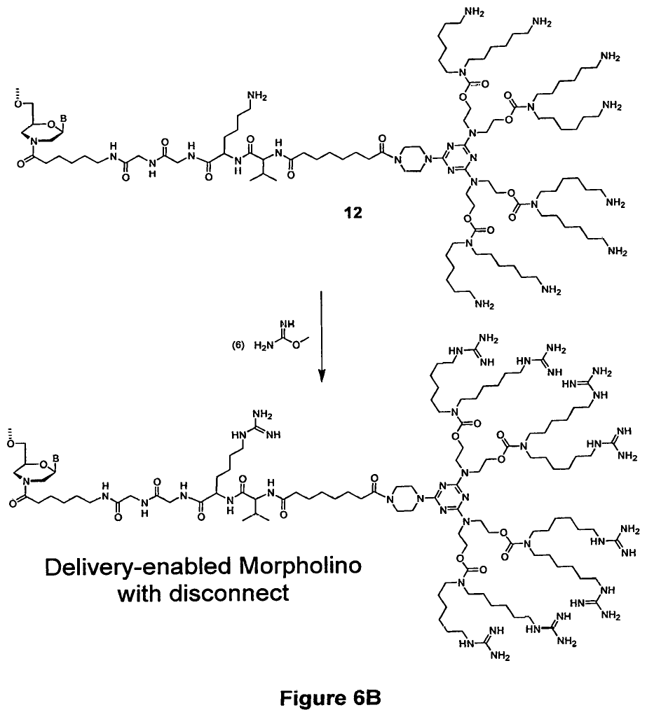

To test these hypotheses, an appropriate structure was synthesized, which comprised: a Morpholino component linked to a disconnect component that was explicitly designed to be rapidly cleaved in acidified lysosomes. In turn, the partially assembled structure was linked to a cationic delivery component to give the structure in FIG. 1c. The disconnect component was synthesized as shown in FIG. 3, and then that disconnect component was covalently inserted between a Morpholino component and the cationic delivery component, as shown in FIG. 6. The molecular structure of the completed delivery-enabled Morpholino with disconnect is shown in FIG. 6B, and the steps in assembly are described in Example 2.

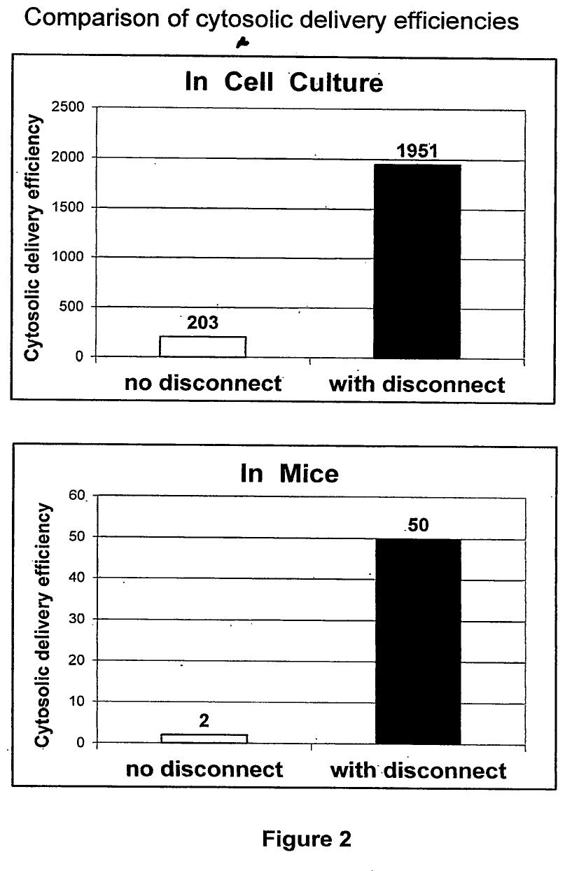

The new structure with covalently-inserted disconnect component (FIG. 1c) was then directly compared to a corresponding structure lacking a disconnect component (FIG. 1b). That comparison entailed assessment of cytosolic delivery efficiency in both cultured cells and in mice (FIG. 2).

As seen in the experimental results shown in FIG. 2, the inserted disconnect component afforded a dramatic increase in cytosolic delivery efficiency relative to the closely-related delivery-enabled Morpholino which lacks the new disconnect component. It is noteworthy that a variety of other properly-designed covalently-inserted disconnect components (FIG. 1c) typically are found to afford about a 1,000% increase in cytosolic delivery efficiency relative to very similar prior art structures lacking a disconnect component (FIG. 1b).

### Key Properties of the 3 Major Components of the Invention:

1) Morpholino Component

The Morpholino component needs to remain intact from its site of introduction into the subject (typically a vein) through to its entry into the cytosol of the cells where it is to carry out its intended blocking of its targeted RNA sequence. It is particularly important that the Morpholino component be resistant to enzymatic degradation during its passage through the acidified lysosome and into the cytosol of the cell.

In regard to this stability requirement, Morpholinos are one of the rare antisense structural types which have the needed resistance to degradation throughout the body, including within acidified lysosomes.

2) Disconnect Component

The disconnect component (an amino acid sequence) is designed to be relatively stable from its site of introduction into the subject until it enters the lysosome of a cell—but upon acidification of that lysosome (a natural process) the disconnect peptide is explicitly designed to be promptly cleaved by acid-activated lysosomal enzymes.

A variety of different targets for that cleavage step can easily be identified from the scientific literature, and then a selected cleavage site can easily be built into the disconnect component.

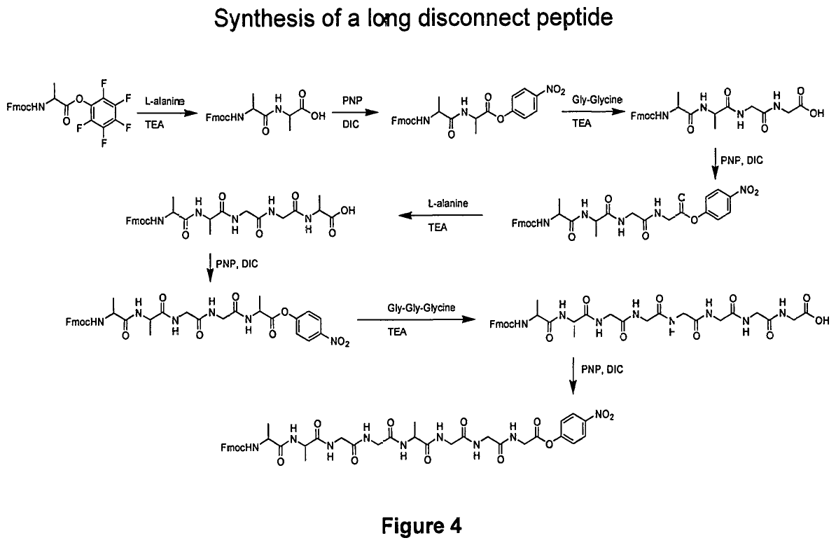

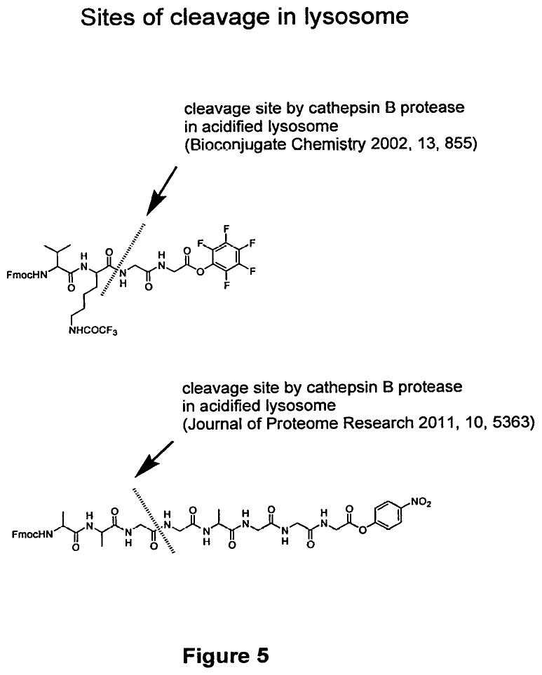

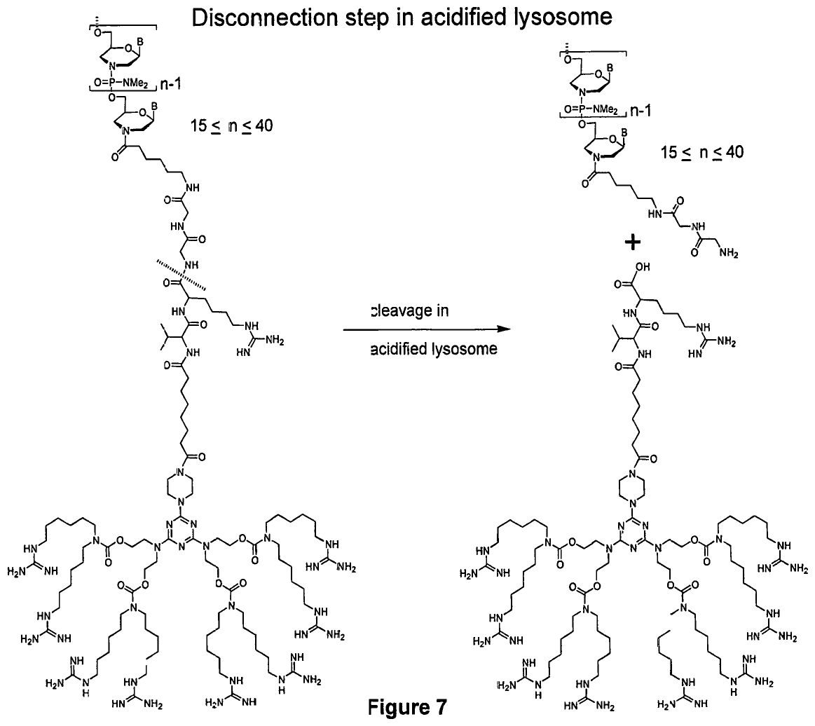

To illustrate, we show two representative disconnect peptides (FIGS. 3 and 4), and then we show in FIGS. 5 and 7 the known preferred sites in the disconnect components which are cleaved in acidified lysosomes (confirmed by mass spectroscopy).

3) Cationic Delivery Component

The cationic delivery component needs to remain intact from its site of introduction into the subject through to its passage into a lysosome of the cell—where it can embed into the inner face of the lysosomal membrane—which apparently partially permeabilizes the lysosomal membrane sufficient for a disconnected Morpholino to slip through into the cytosol of the cell.

The Cationic delivery component is illustrated in FIG. 6B and FIG. 7, and its synthesis is detailed extensively in U.S. Pat. No. 7,935,816. This un-natural dendrimer structure is designed so as to avoid cleavage by lysosomal enzymes—allowing instead for the cationic delivery component to embed into, and partially permeabilize, the lysosomal membrane—thereby providing to the disconnected Morpholino component a tiny but usable path from the partially permeabilized lysosome to the cytosol of the cell.

### Synthesis of a Representative Short Disconnect Component

FIG. 3 shows an overview of synthesis of a representative 4-amino-acid-long disconnect peptide. Example 1 describes that synthesis in greater detail.

### Synthesis of a Representative Longer Disconnect Component

FIG. 4 shows an overview of synthesis of a representative 8-amino-acid-long disconnect peptide.

Assembly of the 3 Major Components into a Completed Product

FIG. 6 shows an overview of the covalent assembly of all three of the major components of a “delivery-enabled Morpholino with disconnect”, where that final product comprises: a Morpholino component linked to a disconnect component which is, in turn, linked to a cationic delivery component. Example 2 describes that assembly process in greater detail.

Use of a “Delivery-Enabled Morpholino with Disconnect”

To use the completed “delivery-enabled Morpholino with disconnect” simply suspend in normal saline, autoclave or sterile filter, and then inject into a vein in the subject to be treated.

### EXAMPLES

### Abbreviations Used in Figures and Examples

Ahx 6-aminohexanoic acid
Ala alanine
Boc t-butoxycarbonyl
DCM dichloromethane
DEA diethylamine
DIC N,N′-diisopropylcarbodiimide
DIPEA N,N-diisopropylethylamine
DMI 1,3-dimethyl-2-imidazolidinone
EA ethyl acetate
Fmoc (9H-fluoren-9-methoxy)carbonyl
Gly glycine
HE hexane
HOBT 1-hydroxybenzotriazole hydrate
hR homoarginine
Lys lysine
PFP pentafluorophenyl
PNP para-nitrophenol
TEA triethylamine
Tfa trifluoroacetyl
TFA trifluoroacetic acid
THE tetrahydrofuran
TLC thin layer chromatography
Val valine

### Example 1 Synthesis of a 4-Amino-Acid-Long Disconnect Peptide (FIG. 3)

### A. Synthesis of Diglycine Derivative (1)

Fmoc-Gly-OPFP (4.63 g, 10 mmol) was dissolved in a mixed solvent system (DCM 100 ml, THF 50 ml). The solution was cooled in an ice bath. Glycine t-butyl ester-HCl (1.68 g, 10 mmol) was added to the solution, followed by DIPEA (5.23 ml, 30 mmol). The mixture was kept at ice bath for 30 min. TLC analysis indicated the reaction was complete (Rf=0.25, EA/HE 1:1). After removal of the solvent, the residue was diluted with DCM (500 ml) and washed with phosphate buffer saline (300 ml) and dried over sodium sulfate. The solution was loaded on a silica gel column (50 g), eluting with DCM (500 ml) and EA/HE 1:2 (1800 ml) to give the purified product 1 (4.1 g).

### B. Synthesis of t-butyl diglycinate (2)

The di-glycine compound 1 was dissolved in acetonitrile (90 ml). Diethylamine (10 ml) was added to the mixture cooled in an ice bath. The solution was kept at room temperature for 1 hour. The volatile materials were removed by evaporation. Hexane (100 ml) was added to remove the dibenzofulvene. The residue containing t-butyl diglycinate (2) was used directly for next step.

### C. Synthesis of tripeptide derivative (4)

Fmoc-Lys(Tfa)-OPFP 3 (10 mmol) and the di-glycine compound 2 (10 mmol) were dissolved in THF (100 ml). DIPEA (3.49 ml, 20 mmol) was added to the mixture cooled in an ice bath. The mixture was kept at 0° C. for 30 min. TLC analysis (EA/HE 3:1) indicated that the reaction was complete. After additional 30 min, the volatile materials were removed by evaporation. The residue was dissolved in DCM and loaded on a silica gel column (50 g), eluting with DCM (500 ml) and EA/IE (3:1, 800 ml) to give the product 4 (4.2 g).

### D. Synthesis of tetrapeptide derivative (5)

The Lys-Gly-Gly compound 4 (4.2 g, 6.62 mmol) was dissolved in THF (180 ml). Diethylamine (20 ml) was added to the solution and the mixture was kept at room temperature for 2 hours. TLC analysis (EA/HE 3:1) indicated that the reaction was complete. Fmoc-Val-OPFP (4 g, 7.91 mmol) was added to the Fmoc-removed intermediate dissolved in THE (100 ml), followed by DIPEA (2.8 ml, 16 mmol). The reaction mixture was kept in an ice bath for 1 hour. After removal of the volatile materials, the product was purified by silica gel column chromatography to give the product 5 (3.6 g).

### E. Synthesis of tetrapeptide activated ester (7)

Fmoc-Val-Lys-Gly-Gly t-butyl ester 5 was treated with TFA (40 ml) to remove the t-butyl ester group to afford the free carboxylic acid 6. The acid 6 (2.4 g) was treated with pentafluorophenol (0.409 ml) and N,N-diisopropylcarbodiimide (0.610 ml) in acetone (100 ml) to give the activated ester 7.

### Example 2 Assembly of a “Delivery-Enabled Morpholino with a Covalently Inserted Disconnect Peptide” (FIG. 6)

### A. Adding Tetrapeptide Moiety to a Morpholino Oligo

To Fmoc-6-aminohexanoic modified Morpholino oligo 8 which was still on the synthesis resin, 20% Piperidine in DMI (0.8 mL) was added to the column and kept at room temperature for 2 minutes. Same amount of piperidine was added to the column and kept at room temperature for 2 minutes. The column was washed with DMI (1 mL×2) to remove the excess reagent. Tetrapeptide 7 (Fmoc-Val-(Tfa)Lys-Gly-Gly-OPFP) (55 mg) and HOBt (25 mg) were dissolved in 5% t-butyldiethanolamine in DMI (420 microL). The solution was added to the above Morpholino oligo column (1000 nanomoles) and the mixture was heated at 50° C. for 2 hours. The column was then washed with DMI (1 mL×2) to remove the excess reagent to give intermediate 9. 20% Piperidine in DMI (0.8 mL) was added to the column and kept at room temperature for 2 minutes. Same amount of piperidine was added to the column and kept at room temperature for 2 minutes. The column was washed with DMI (1 mL×2) to remove the excess reagent, ready for next reaction step.

### B. Adding Pre-Cationic Delivery Component (10)

Pre-cationic delivery component 10 (0.13M, 250 microL) was mixed with HOBt (12.5 mg) and N-methylmorpholine (12.5 microL). The mixture was added to the above column and the column was heated at 60° C. for 4 hours. The column was washed with DMI (4 mL) to remove excess reagent to give intermediate 11.

### C. Deprotecting and Converting Amino Groups to Guanidines

The above column was rinsed with acetonitrile (4 mL). After solvent was removed, the resin was taken out and put in a vial. Concentrated ammonia (350 microL) was added to the vial and the mixture was incubated at 53° C. for 6 hours to generate the intermediate 12. Additional ammonia (350 microL) was added to the above mixture while cooled. A solution of O-methylisourea hydrochloride (200 mg) in water (200 microL) was added to the mixture. The mixture was incubated at 50° C. for 45 minutes. The vial was cooled in an ice bath and water (0.7 mL) was added to dilute the mixture.

### D. Isolating Delivery-Enabled Morpholino with Disconnect

The Oasis cartridge was conditioned with methanol (5 mL), followed by water (5 mL). The mixture prepared above was loaded to the cartridge. Water (0.5 mL) was used to wash the vial and the washing was also loaded on to the cartridge. The cartridge was washed with water (20 mL), followed by phosphate buffer (0.05M, pH 7.0, 2 mL), and again with water (5 mL). 50% acetonitrile (8 mL) was used to elute the final product, delivery-enabled Morpholino with disconnect. The solvents were removed by freeze-drying.

1. A composition for efficient delivery of Morpholino antisense oligos into the cytosol of living cells, which may be in a living subject, wherein the composition includes:
i) a Morpholino antisense oligo;
ii) a disconnect peptide that is effective to be cleaved within a lysosome of the cell is covalently linked to the Morpholine antisense oligo; and,
iii) a cationic dendrimer is covalently linked to the disconnect peptide.

2. The composition of claim 1, wherein the antisense oligo is not less than and not more than 40 nucleobase subunits long.

3. The composition of claim 1, wherein the disconnect peptide has the sequence: Val-Lys-Gly-Gly.

4. The composition of claim 1, wherein the disconnect peptide has the sequence: Val-hR-Gly-Gly.

5. The composition of claim 1, wherein the disconnect peptide has the sequence: Ala-Ala-Gly-Gly-Ala-Gly-Gly-Gly.

6. The composition of claim 1, wherein the cationic dendrimer contains at least 6 positive charges and not more than 12 positive charges.

7. The composition of claim 1, wherein the antisense oligo is about 25 subunits long, the disconnect peptide has the sequence: Val-Lys-Gly-Gly; and, the cationic dendrimer contains 8 positive charges.

8. A composition for efficient delivery of Morpholino antisense oligos into the cytosol of living cells, wherein the composition includes the structure below: [chemical structure]
